(19) United States
(12) Patent Application Publication
(10) Pub. No.: US 2010/0316716 A1
(43) Pub. Date: Dec. 16, 2010

(54) METHOD FOR TREATING HERPES ZOSTER LESIONS

(76) Inventor: Abdul R. Abukurah, Northridge, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/789,011

(22) Filed: May 27, 2010

Related U.S. Application Data
(60) Provisional application No. 61/186,261, filed on Jun. 11, 2009.

(51) Int. Cl. A61K 33/06; A61P 31/12; A61K 9/00; A61K 33/00

(52) U.S. Cl. 424/484; 424/682; 424/720

(57) ABSTRACT

The present invention relates to the treatment of herpes zoster lesions. Compositions comprising an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride are described as well as methods of their use for treatment purposes.

### CROSS-REFERENCE TO RELATED APPLICATION

This application claims the benefit of U.S. Provisional Application No. 61/186,261, filed Jun. 11, 2009, which is incorporated herein by reference in its entirety.

### BACKGROUND

Herpes zoster (“shingles”) is a viral revival of the chicken pox virus that lies dormant in the skin and mucous membranes of human beings. If the immunity of a person carrying the varicella zoster virus is decreased—such as due to malnourishment, chronic disease, or cancer—the virus can reactivate and appear in the form of lesions or blisters, causing pain and irritation discomfort.

Current treatments for shingles include medication for treating pain (e.g., pills or topical treatments) as well as therapeutic treatment using a pharmaceutical, such as acycloguanosine. Using current treatment regimens, shingles typically take four to six weeks to heal.

Given the acute discomfort of those afflicted with shingles, a treatment that improves the time required to treat the condition is desirable.

### SUMMARY

Presented herein are compounds, compositions, and methods for treating herpes zoster lesions. In some embodiments, a composition for topical application to a herpes zoster lesion is provided, comprising: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound. Also provided is a composition for topical application to a herpes zoster lesion, consisting essentially of: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound.

Other embodiments contemplate a method for treating of a herpes zoster lesion in a subject in need of treatment, comprising topically administering to said lesion a composition comprising: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound.

In a further embodiment, the present invention contemplates a method for treating of herpes zoster lesions in a subject in need of treatment, consisting essentially of topically administering to said lesion a composition consisting essentially of: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a photograph of a portion of a patient's skin covered in shingles;

FIG. 2 is a photograph of the same portion of the patient's skin as pictured in FIG. 1 after three days of treatment using the embodiments described herein;

FIG. 3 is a photograph of a portion of a patient's skin covered in shingles; and

FIG. 4 is a photograph of the same portion of the patient's skin as pictured in FIG. 3 after three days of treatment using the embodiments described herein.

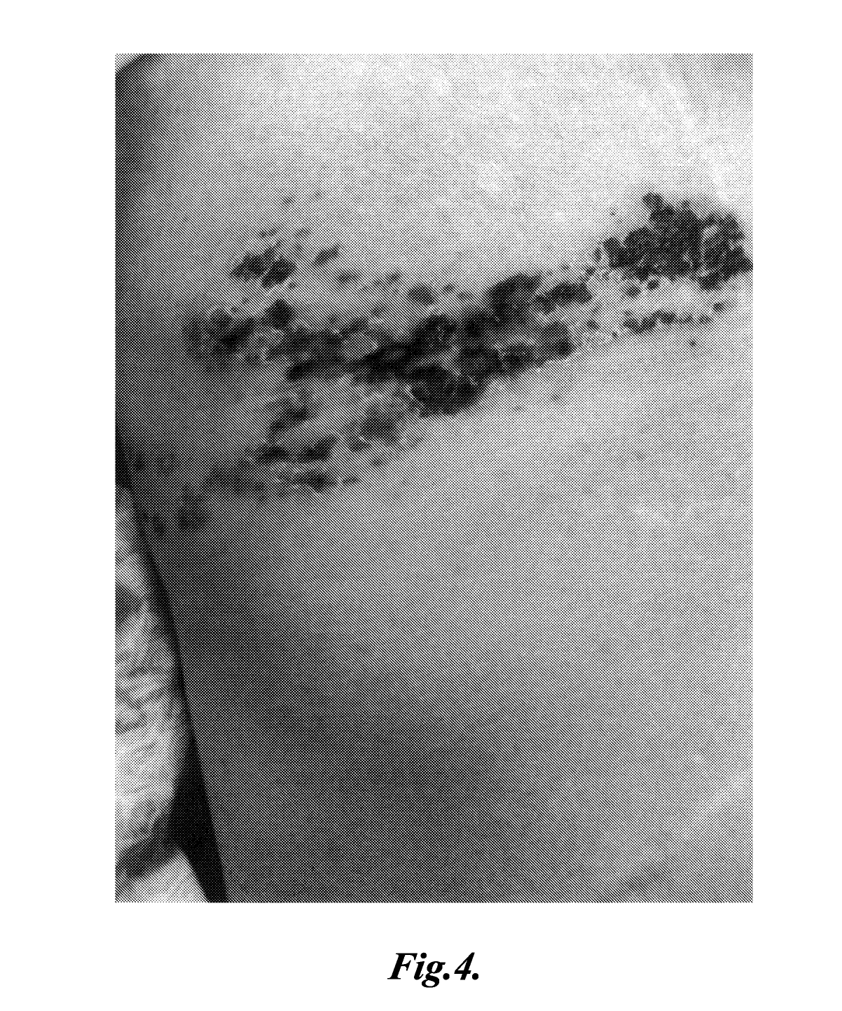

### DETAILED DESCRIPTION

Compositions and methods are provided for the treatment of herpes zoster lesions (“shingles”). Treatment of shingles using the provided embodiments typically begins to alleviate the pain and irritation of shingles within one to two days of application. Additionally, shingles will typically begin to shrink in size and disappear more expeditiously than the four to six weeks required for known treatments. Persons of skill in the art are familiar with methods of identifying herpes zoster lesions.

Accordingly, some embodiments provide a composition for topical application to a herpes zoster lesion, comprising: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound. Some embodiments contemplate a composition for topical application to a herpes zoster lesion, consisting essentially of: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound.

Methods of using compounds and compositions described herein are also contemplated, such as a method for treating of a herpes zoster lesion in a subject in need of treatment, comprising topically administering to said lesion a composition comprising: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound.

Any composition described herein may comprise potassium aluminum sulfate dodecahydrate. Any composition may comprise water. In some embodiments, the active compound in a composition is potassium aluminum sulfate dodecahydrate and the matrix comprised in the composition comprises water.

A composition may be in the form of a solution, such as an aqueous solution, or any other formulation described herein. A solution may be administered in spray form, for example. Following administration, a composition may then be left to air dry. A composition may be administered twice daily for multiple days, or any other timeframe described herein.

In some embodiments, a method for treating of herpes zoster lesions in a subject in need of treatment is provided that consists essentially of topically administering to said lesion a composition consisting essentially of: an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and a matrix capable of supporting the compound.

U.S. application Ser. No. 12/575,232, entitled, “Methods for Treating Skin Lesions,” is also referenced and is incorporated herein by reference in its entirety.

### Active Compound(s)

It has been found that topical application of particular active compounds can provide rapid relief and therapeutic benefit to individuals suffering from herpes zoster. An active compound may be an alum, which is a class of compounds well-known in the art (e.g., compounds having the stoichiometry of AB(SO₄)₂.12H₂O, where A may be, e.g., potassium or ammonium, and B may be aluminum), or a similar astringent. Suitable active compounds include potassium aluminum sulfate dodecahydrate (KAl(SO₄)₂.12H₂O), sometimes referred to as potassium alum or potash alum, and ammonium aluminum sulfate dodecahydrate (NH₄Al(SO₄)₂.12H₂O). Anhydrous potassium aluminum sulfate and anhydrous ammonium aluminum sulfate may alternatively be used. Ammonium chloride, which may be considered an anhydrous form of ammonium, may be employed. Combinations of these active compounds may be used as well. Aluminum salt suppliers are well-known in the art (e.g., Hospira, Inc., or Abbott Laboratories).

As used herein, the term “effective” (e.g., “an effective amount”) means adequate to accomplish a desired, expected, or intended result. For example, an effective amount of an active compound may refer to an amount of active compound necessary to reduce the appearance of a herpes zoster lesion, or reduce pain associated with such a lesion.

### Additional Ingredients, Formulations, and Packaging

Because the active compounds typically occur in the solid state at standard temperature and pressure, a composition comprising an active compound may include a matrix in which the active compound is supported for convenient application to a patient's lesion(s). As used herein with reference to the matrix, the term “supported” means that the active compound is either dissolved or suspended in the matrix. Representative matrices include liquids (i.e., a solution), gels, and creams. A composition including a matrix may be topically applied to a patient's shingles. Suitable matrices will not inhibit the efficacy of the active compound for treating shingles, and will allow for convenient application to affected areas of skin.

In one embodiment, the matrix is a liquid that is a suitable solvent for dissolving an active compound to form a solution that is typically homogeneous. An exemplary solvent is water. The active compound is typically dissolved in the solvent at a concentration suitable to provide efficacy upon application of the solution to shingles. A representative concentration for a solution is an amount of active compound dissolved in an amount of solution so as to provide a solution at about 35-50% saturation. In some embodiments, the amount of active compound provides a solution at about, at least about, or at most about 35%, 36%, 37%, 38%, 39%, 40%, 41%, 42%, 43%, 44%, 45%, 46%, 47%, 48%, 49%, or 50% saturation, or any range derivable therein.

Methods of formulation are well known in the art and are disclosed, for example, in Remington: The Science and Practice of Pharmacy, Mack Publishing Company, Easton, Pa., 19th Edition (1995). Pharmaceutical or pharmaceutically acceptable formulations for topical administration of a composition include ointments, pastes, creams, lotions, gels, powders, solutions, sprays, inhalants, or patches. The phrase “pharmaceutically acceptable” refers to molecular entities and compositions that do not produce an adverse, allergic, or other untoward reaction when administered to a patient.

An active compound is typically admixed under sterile conditions with other ingredients as described herein. The ointments, pastes, creams, gels, etc., may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suitable formulations will not inhibit the efficacy of the active compound for treating shingles, and will allow for convenient application to affected areas of skin.

In some embodiments, an active compound is combined with a personal, biologically inert lubricant such as K-Y Jelly®, and applied to a lesion.

A product including a composition of the present invention may be held in a collapsible container as a liquid, cream, lotion, ointment, or gel form. The package of the product may bear the warning such as “do not use this product on or around the eye in any form or shape.”

### Administration

As noted above, an active compound or a composition comprising an effective amount of an active compound may be topically administered. In some embodiments, a composition comprising an active compound is a solution. The solution may be topically applied directly to the herpes zoster lesion using a suitable application method, such as by a spray bottle. A representative dose of a solution for a typical lesion is about 25-50 mL at the saturation concentrations described herein. In some embodiments, the dose is about, at most about, or at least about 25, 26, 27, 28, 29, 30, 31, 32, 33, 34, 35, 36, 37, 38, 39, 40, 41, 42, 43, 44, 45, 46, 47, 48, 49, or 50 mL, or any range derivable therein. The solution is then preferably allowed to air dry after topical application to the lesion.

The active compound or composition may be applied to the lesions one or more times per day for one or more successive days. The frequency of application depends on the efficacy of the active compound on a patient's lesions. In an exemplary regimen for treatment, a composition comprising an effective amount of an active compound is applied to the lesion twice per day for one to two days, after or during which the lesion begins to heal. The associated pain and irritation of the lesion is typically also reduced during this time. In some embodiments, a treatment time lasts about, at most about, or at least about 1, 2, 3, 4, 5, 6, or 7 days, or any range derivable therein.

### Additional Definitions

As used herein, the term “patient” or “subject” refers to a living mammalian organism, such as a human.

“Treatment” and “treating” as used herein refer to administration or application of an active compound to a patient or performance of a procedure or modality on a patient for the purpose of obtaining a therapeutic benefit of a disease or health-related condition. For example, a patient having a herpes zoster lesion may be subjected to a treatment comprising administration of a composition described herein in order to reduce the appearance of the lesion or to minimize conditions associated with the lesion, such as irritation or pain.

The term “therapeutic benefit” as used throughout this application refers to anything that promotes or enhances the well-being of the patient with respect to the medical treatment of a condition. This includes, but is not limited to, a reduction in the onset, frequency, duration, or severity of the signs or symptoms of a herpes zoster lesion.

In any embodiment herein, the term “comprising” may be substituted with “consisting essentially of” or “consisting of.”

For those embodiments reciting “consisting essentially of,” it is noted that non-limiting examples of materials and steps that do not materially affect the basic and novel aspects of the compositions and methods described herein include those that do not change the chemical structure of the active compound employed, that do not interfere with access of the active compound to the lesion, or those that do not decrease the effective amount of the active compound that is administered. Any further additional ingredient described herein (e.g., a matrix) may be combined in a composition that comprises, consists essentially of, or consists of one or more active compounds.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

As used herein, “a” or “an” means one or more, unless clearly indicated otherwise.

Throughout this application, the term “about” is used to indicate that a value includes the standard deviation of error for the device and/or method being employed to determine the value.

### Examples

In trials with the disclosed compound topical application of 30 cc's of a 50% saturated potassium aluminum sulfate dodecahydrate solution by use of a spray bottle to the herpes zoster lesions caused some minor stinging sensation for a brief period, approximately 2-3 minutes. The sprayed solution was allowed to air dry.

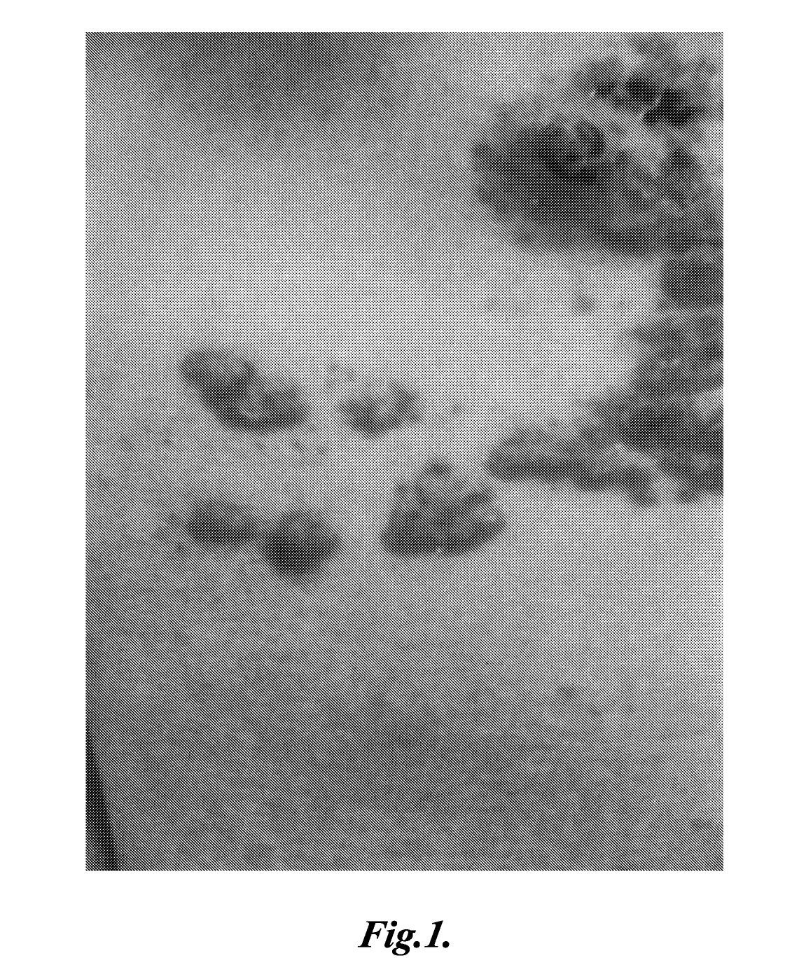

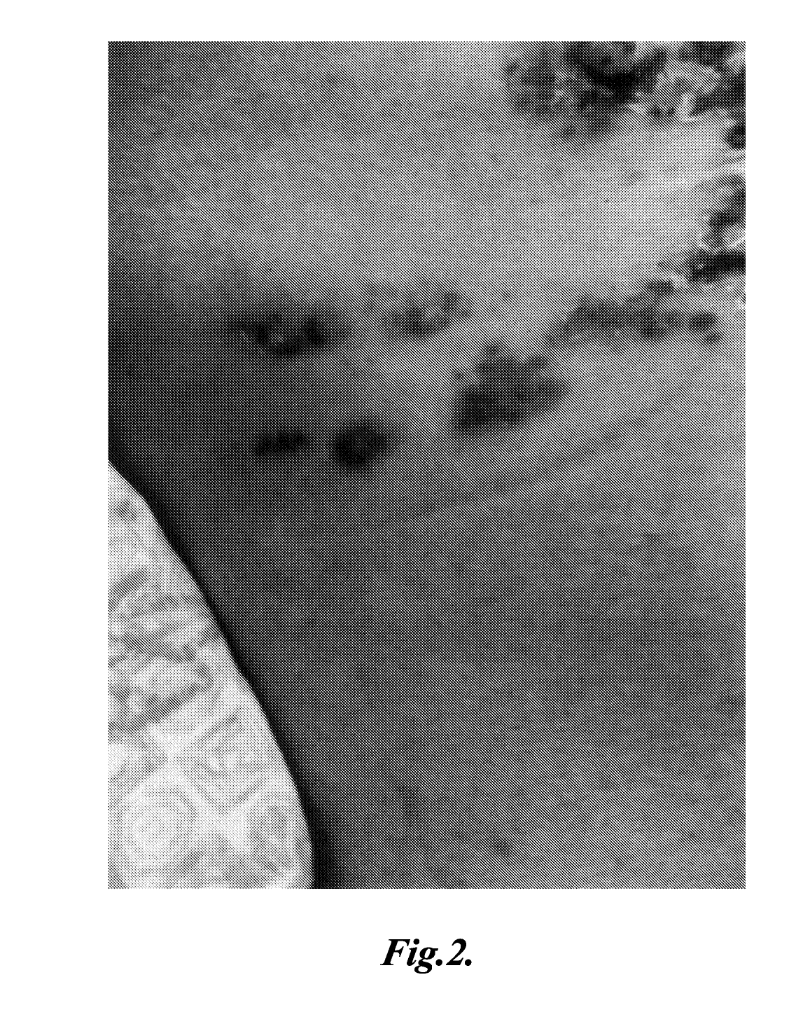

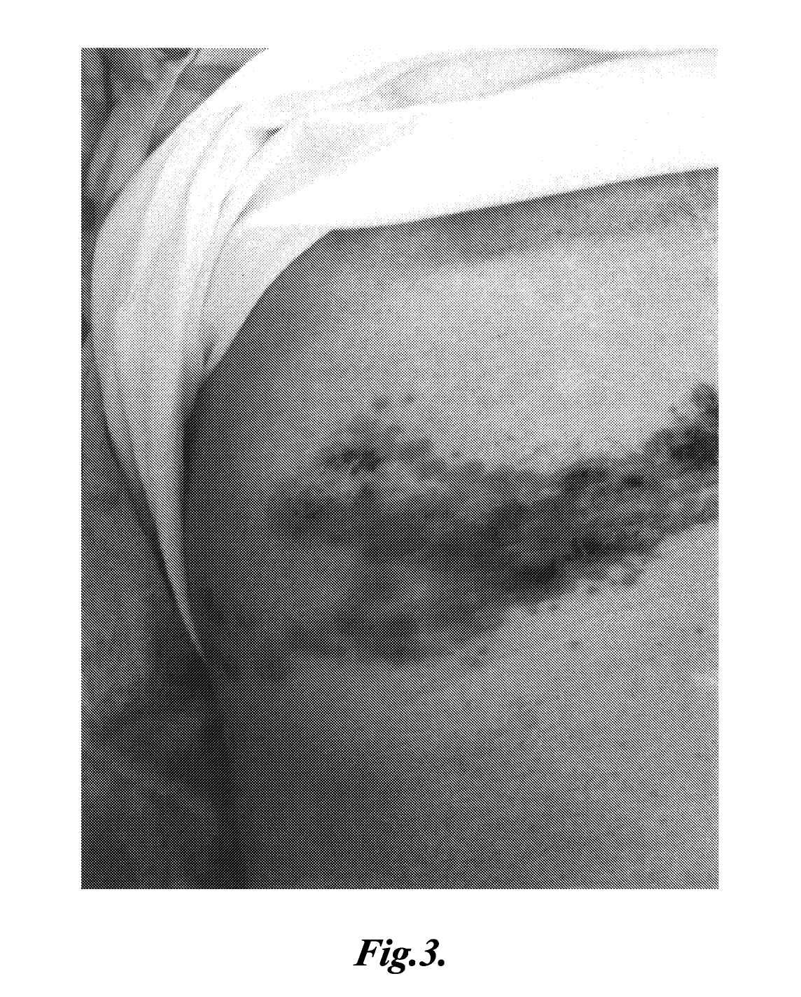

FIG. 1 shows herpes zoster lesions prior to treatment on the chest of a patient with chronic kidney disease. FIG. 2 shows the same lesion after only three days of twice-daily topical application of aluminum potassium sulfate dodecahydrate dissolved in water at 50% saturation. The lesions have receded significantly and are crusting, which indicates healing. Before and after photographs of another set of lesions on the patient are shown in FIGS. 3 (before) and 4 (after). Similar positive results are shown.

While illustrative embodiments have been illustrated and described, it will be appreciated that various changes can be made therein without departing from the spirit and scope of the invention.

The embodiments of the invention in which an exclusive property or privilege is claimed are defined as follows:

1. A composition for topical application to a herpes zoster lesion, comprising:
an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and
a matrix capable of supporting the compound.

2. A composition for topical application to a herpes zoster lesion, consisting essentially of:
an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and
a matrix capable of supporting the compound.

3. A method for treating of a herpes zoster lesion in a subject in need of treatment, comprising topically administering to said lesion a composition comprising:
an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and
a matrix capable of supporting the compound.

4. The method of claim 3, wherein the composition comprises potassium aluminum sulfate dodecahydrate.

5. The method of claim 3, wherein the compound is potassium aluminum sulfate dodecahydrate and the matrix comprises water.

6. The method of claim 3, wherein the composition is administered in a spray form.

7. The method of claim 6, further comprising the step of allowing the administered composition to air dry.

8. The method of claim 3, wherein the composition is administered twice daily for about 1-4 days.

9. A method for treating of herpes zoster lesions in a subject in need of treatment, consisting essentially of topically administering to said lesion a composition consisting essentially of:
an effective amount of a compound selected from the group consisting of potassium aluminum sulfate, potassium aluminum sulfate dodecahydrate, ammonium aluminum sulfate, ammonium aluminum sulfate dodecahydrate, and ammonium chloride, and combinations thereof; and
a matrix capable of supporting the compound.
